(19) United States
(12) Patent Application Publication
(10) Pub. No.: US 2014/0179975 A1
(43) Pub. Date: Jun. 26, 2014

(54) SPLIT-SHELL RAFFINATE COLUMNS AND METHODS FOR USE IN CONTINUOUS ADSORPTIVE SEPARATION PROCESSES

(71) Applicant: UOP LLC, Des Plaines, IL (US)

(72) Inventors: Soumendra Banerjee, New Delhi (IN); Edwin M. Victor, Arlington Heights, IL (US); Richard Hoehn, Mt. Prospect, IL (US); Bruce Richard Beadle, Kildeer, IL (US)

(73) Assignee: UOP LLC, Des Plaines, IL (US)

(21) Appl. No.: 13/727,284

(22) Filed: Dec. 26, 2012

(51) Int. Cl. B01D 3/32; C07C 7/12

(52) U.S. Cl. CPC B01D 3/32; C07C 7/12
USPC 585/826; 202/158

(57) ABSTRACT

A split-shell column includes a raffinate column portion for separating a raffinate material from a desorbent material and a desorbent rerun column portion for separating heavy contaminants from the desorbent material. A feed to the desorbent rerun column portion is provided from the desorbent material in the raffinate column. The desorbent rerun column portion occupies a portion of a lower end of the split-shell column and is thermally separated from the raffinate column portion.

### TECHNICAL FIELD

The present disclosure relates to a continuous adsorptive separation process for separating chemical compounds such as C₈ aromatic hydrocarbons and equipment for use therein. The present disclosure specifically relates to split-shell raffinate columns and methods for use in continuous adsorptive separation processes.

### BACKGROUND

In many commercially important petrochemical and petroleum industry processes, it is desirable to separate closely boiling chemical compounds or to perform a separation of chemical compounds by structural class. It is very difficult or impossible to do this by conventional fractional distillation due to the requirement for numerous fractionation columns that may consume excessive amounts of energy. The relevant industries have responded to this problem by utilizing other separatory methods that are capable of performing a separation based upon chemical structure or characteristics. Adsorptive separation is one such method and is widely used to perform these separations.

In the practice of adsorptive separation, a feed mixture including two or more compounds of different molecular structure is passed through one or more beds of an adsorbent that selectively adsorbs a compound of one molecular structure while permitting other components of the feed stream to pass through the adsorption zone in an unchanged condition. The flow of the feed through the adsorbent bed is stopped, and the adsorption zone is then flushed to remove non-adsorbed materials surrounding the adsorbent. Thereafter the desired compound is desorbed from the adsorbent by passing a desorbent stream through the adsorbent bed. The desorbent material is commonly also used to flush non-adsorbed materials from the void spaces around and within the adsorbent. This could be performed in a single large bed of adsorbent or in several parallel beds on a swing bed basis. However, it has been found that simulated moving bed adsorptive separation provides several advantages such as high purity and recovery. Therefore, many commercial scale petrochemical separations, especially for specific paraffins and xylenes, are performed using countercurrent simulated moving bed (SMB) technology.

Industrial scale simulated moving bed systems require numerous adsorbent beds, columns, and other support equipment to process the volume of feed mixture required in commercial applications. Each piece of equipment in the system adds an expense, both in terms of capital costs and operational costs. As such, it is desirable to reduce the number of individual components in the SMB system by combining equipment functionalities wherever possible.

Accordingly, it is desirable to provide improved apparatus for use with continuous adsorptive separation processes. Furthermore, other desirable features and characteristics of the inventive subject matter will become apparent from the subsequent detailed description of the inventive subject matter and the appended claims, taken in conjunction with the accompanying drawings and this background of the inventive subject matter.

### BRIEF SUMMARY

Disclosed herein, in one exemplary embodiment, a split-shell column includes a raffinate column portion for separating a raffinate material from a desorbent material and a desorbent rerun column portion for separating heavy contaminants from the desorbent material. A feed to the desorbent rerun column portion is provided from the desorbent material separated in the raffinate column. The desorbent rerun column portion occupies a portion of a lower end of the split-shell column and is thermally separated from the raffinate column portion.

In another exemplary embodiment, a method for separating hydrocarbon mixtures includes directing a raffinate stream into a split-shell column, the raffinate stream including a raffinate material and a desorbent material, the split-shell column including a raffinate column portion and a desorbent rerun column portion and separating the raffinate material from the desorbent material in the raffinate column portion of the split-shell column. The method further includes directing a first portion of the desorbent material separated in the raffinate column portion to the desorbent rerun column portion, separating the desorbent material into decontaminated desorbent material and heavy contaminants, and directing the decontaminated desorbent material back into the raffinate column portion.

In yet another exemplary embodiment, a split-shell column includes a raffinate column portion for separating a raffinate material from a desorbent material. The raffinate column portion extends from a bottom of the split-shell column to a top of the split-shell column. The split-shell column further includes a desorbent rerun column portion for separating heavy contaminants from the desorbent material. The desorbent rerun column portion extends from the bottom of the split-shell column but does not extend to the top of the split-shell column. A feed to the desorbent rerun column portion is provided from the desorbent material separated in the raffinate column. The desorbent rerun column portion occupies a portion of a lower end of the split-shell column and is thermally separated from the raffinate column portion with an insulated dividing wall. The desorbent rerun column portion includes a chimney disposed through a blind tray. Further, the desorbent material separated in the desorbent rerun column portion is directed back into the raffinate column portion through the chimney disposed through the blind tray.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE FIGURES

The various embodiments will hereinafter be described in conjunction with the following drawing figures, wherein like numerals denote like elements, and wherein:

FIG. 1 is a schematic representation of an for use with a continuous adsorptive separation process as is known in the prior art; and

FIG. 2 is a cross-sectional view of an exemplary split-shell combined raffinate column in accordance with an embodiment of the present disclosure.

### DETAILED DESCRIPTION

The following detailed description is merely exemplary in nature and is not intended to limit the invention or the application and uses of the invention. All of the embodiments and implementations of the split-shell columns described herein are exemplary embodiments provided to enable persons skilled in the art to make or use the invention and not to limit the scope of the invention, which is defined by the claims. Furthermore, there is no intention to be bound by any expressed or implied theory presented in the preceding technical field, background, brief summary, or the following detailed description.

In many commercially important petrochemical and petroleum industry processes it is desirable to separate chemical compounds that have boiling point temperatures that are within few degrees of each other, referred to in the art as “closely boiling” compounds, or to perform a separation of chemical compounds by structural class. Examples of this are the recovery of normal paraffins from petroleum kerosene fractions for use in the production of detergents and the recovery of para-xylene from a mixture of C₈ aromatics with the para-xylene being used in the production of polyesters and other plastics. Meta-xylene is also recovered by adsorptive separation from xylene feed mixtures. A “feed mixture” is a mixture containing one or more extract components and one or more raffinate components to be separated by the process. The term “feed stream” indicates a stream of a feed mixture that is passed into contact with the adsorbent used in the process. The separation of high octane hydrocarbons from a naphtha boiling range petroleum fraction and the recovery of olefins from a mixture of paraffins and olefins are other examples of situations in which the close volatility of the compounds makes the use of fractional distillation impractical. Adsorptive separation of different classes or types of compounds is performed using adsorptive separation when there is an overlap in boiling points across a broad boiling range of compounds. For instance, in the case of the recovery of the normal paraffins referred to above, it is often desirable to recover paraffins having a range of carbon numbers extending from about C₉ to C₁₂. This would require at least one fractional distillation column for each carbon number. The resulting capital and operating costs make separation by fractional distillation economically unfeasible.

The relevant industries have responded to this problem by utilizing other reparatory methods that are capable of performing a separation based upon chemical structure or characteristics. Adsorptive separation is often the method of choice and is widely used to perform the separations mentioned above. In adsorptive separation, one or more compounds are selectively retained upon an adsorbent and then released by the application of a driving force for the desorption step. The driving force may be heat or a reduced pressure. In the subject process, this driving force is provided by contacting the “loaded” adsorbent (i.e., having the selected compounds retained thereon) with a desorbent compound. Therefore, the adsorbent must be continuously cycled between exposure to the feed stream and a stream including the desorbent. As described below, this forms at least two effluent streams; the raffinate stream, which contains un-adsorbed compounds, and the extract stream, containing the desired adsorbed compounds. Both streams also include the desorbent compound. It is necessary to remove the desorbent from these streams to purify them and also to recover the desorbent for re-use. An “extract component” is a compound or class of compounds that is more selectively adsorbed by the adsorbent while a “raffinate component” is a compound or type of compound that is less selectively adsorbed. The term “desorbent material” generally refers to a material capable of desorbing an extract component from the adsorbent. The term “raffinate stream” or “raffinate output stream” means a stream in which a raffinate component is removed from the adsorbent bed after the adsorption of extract compounds. The composition of the raffinate stream can vary from essentially 100% desorbent material to essentially 100% raffinate components. The term “extract stream” or “extract output stream” means a stream in which an extract material, which has been desorbed by a desorbent material, is removed from the adsorbent bed. The extract stream may be rich in the desired compound or may only contain an increased concentration. The term “rich” is intended to indicate a concentration of the indicated compound or class of compounds greater than 50 mol-% and preferably above 75 mol-%. The composition of the extract stream can vary from essentially 100% desorbent material to essentially 100% extract components.

The various embodiments contemplated herein provide a more economical process for recovering the desorbent compound from the extract and raffinate streams produced during adsorptive separation. In addition, they provide an improved simulated moving bed adsorptive separation process having reduced capital costs. These improvements are provided as a result of consolidating two separate columns (as in the prior art) into a single, split-shell column. This reduces the number of columns required, thus reducing capital costs, and further allows for shared utility inputs (heating, cooling, etc.) into the single, split-shell column, thus providing for more economical operation.

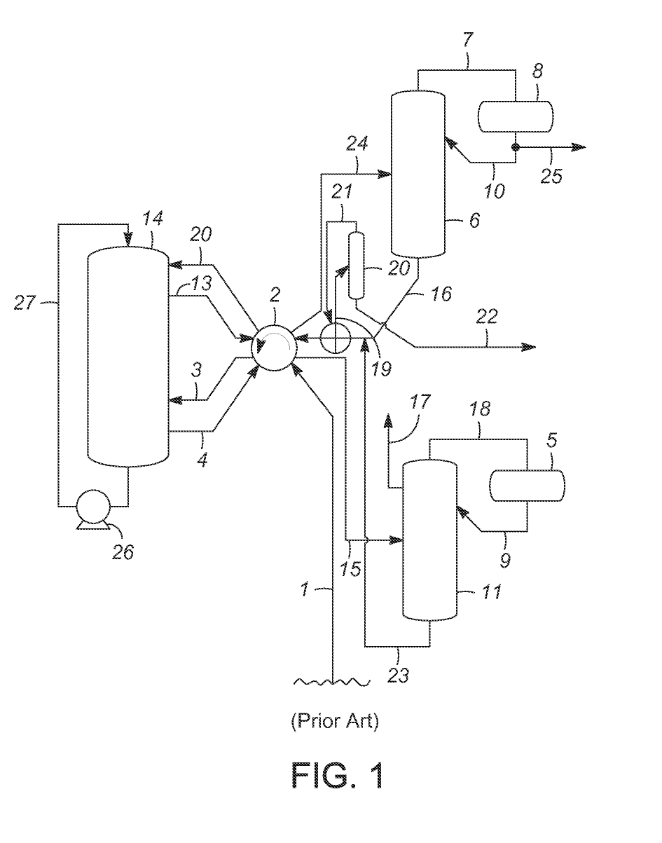

The overall operation of a conventional adsorptive separation process may be discerned by reference to FIG. 1, which illustrates a simulated moving bed adsorptive separation process having a single adsorbent chamber 14 and a single fractional distillation column 6. For purposes of description, it is assumed that the process is being employed to separate the feed stream of line 1 including a mixture of several C₈ aromatic hydrocarbons including para-xylene, meta-xylene, ortho-xylene, and ethylbenzene. The very close volatilities of these compounds make it impractical to separate them on a commercial scale by fractional distillation. Therefore the predominant commercial reparatory techniques are crystallization and adsorptive separation. In the process depicted in FIG. 1, the feed stream of line 1 is passed into a rotary valve 2. This rotary valve has a number of ports (openings) corresponding to the number of adsorption chamber process streams plus the number of “bed lines” for connecting to each sub bed of adsorbent located in the one or more adsorbent chambers used in the process. As the adsorbent chamber(s) may contain from about 8 to about 24 adsorbent sub beds, there are a large number of bed lines involved in the process. For simplicity only those four bed lines in use at the moment in time being depicted are shown in FIG. 1.

The rotary valve 2 directs the feed stream into a bed line 3, which carries it to the adsorbent chamber 14. The feed stream enters into the adsorbent chamber at a boundary between two of the sub beds (not shown) and is distributed across the cross-section of the chamber. It then flows downward or downstream through several sub-beds of adsorbent containing particles. The terms “upstream” and “downstream” are used herein in their normal sense and are interpreted based upon the overall direction in which liquid is flowing in the adsorbent chamber. That is, if liquid is generally flowing downward through a vertical adsorbent chamber, then upstream is equivalent to an upward or higher location in the chamber. The quantity of adsorbent in these beds selectively retains one compound or structural class of compound, which in this instance is para-xylene. The other components of the feed stream continue to flow downward and are removed from the adsorbent chamber in the raffinate stream carried by line 4. The raffinate stream will also include a varying amount of desorbent compound(s) flushed from the inter-particle void volume and removed from the adsorbent itself. This desorbent is present in the bed prior to the adsorption step due to the performance of the desorption step.

The raffinate stream enters the rotary valve 2 and is then directed into line 15. Line 15 carries the raffinate stream to a raffinate column 11. This raffinate column 11 contains, for example, about 30 fractionation trays or more.

During operation, the raffinate stream 15 entering the raffinate column 11 is separated, with the less volatile desorbent component(s) moving downward out of the fractionation zone and emerging into the lower portion of the column 11 and leave(s) the column via line 23. The more volatile raffinate components, e.g. meta- and ortho-xylene, of the feed stream are concentrated into an overhead vapor stream and removed from the raffinate column 11 via line 18. This stream is passed through an overhead condenser (not shown) and the resultant fluid is passed into an overhead receiver 5. The collected overhead liquid is withdrawn from the receiver and returned via line 9 to the column 11 as a reflux stream. Uncondensed gases may be removed by a line (not shown). The raffinate components are removed in the side-cut stream of line 17 and passed to a xylene isomerization zone to produce more para-xylene. This overhead arrangement is used to dry the raffinate stream, with water (not shown) being drained from the receiver 5.

Simultaneously, a stream of desorbent is passed into the adsorbent chamber 14 at a different inlet point via line 20. As the desorbent moves downward through the selective adsorbent, it removes para-xylene from the adsorbent in a section of the chamber used as the desorption zone. This creates a mixture of para-xylene and desorbent that flows through the section of the adsorbent chamber 14 functioning as the desorption zone. As part of this flow, this mixture is removed from the bottom of the chamber 14 and returned to the top of the chamber via a line 27, which is referred to in the art as the “pump-around line,” with pump 26 providing the pumping power therefor. The liquid then flows through more sub-beds of adsorbent at the top of the chamber and is removed from the adsorbent chamber 14 via line 13 as the extract stream. This stream is passed into the rotary valve 2. The rotary valve directs the extract stream of line 13 into line 24. Line 24 delivers the extract stream into an extract column 6.

Like the raffinate column 11, the extract column 6 contains a number of fractionation trays extending across the column. The more volatile extract component, primarily para-xylene, moves upward through the extract column 6 and is removed from column 6 via line 7 in an overhead vapor stream. If present in the feed, toluene will, to some extent, co-adsorb and be present in the extract. It can be removed downstream in a finishing column. This second overhead vapor stream is passed through an overhead condenser (not shown) and then into a second overhead receiver 8. The liquid collected in this second receiver is divided into a reflux stream returned to the top of the extract column 6 via line 10 and an extract product stream removed from the process via line 25. As with the first fractionation zone, the lower end of the second zone is in open communication with the column 6.

The desorbent compound(s) present in the extract stream of line 24 is driven downward in the extract column 6. The desorbent enters the lower portion of the extract column 6 and falls upon the trays as it enters the bottom portion of the column 6. A stream of the desorbent is removed from this storage volume in the bottom of the column via line 16 and then is passed into the rotary valve 2.

From either the raffinate column 11 or the extract column 6, any heavy (i.e., C₉+) contaminants that were originally present in the feed line 1 will accumulate in the desorbent. If not removed, these heavy species would tend to reduce the effectiveness of the adsorbent. In order to prevent this accumulation, provision is made to take a slip-stream of the recycled desorbent, via line 19, to a small desorbent rerun column 20 where any heavy contaminants are rejected via line 22. The de-contaminated desorbent returns via line 21 to rejoin the recycled desorbent stream prior to its reintroduction into the rotary valve 2. As such, the configuration of the SMB system shown in FIG. 1 requires a separate desorbent rerun column to prevent the accumulation of heavy contaminants in the desorbent stream.

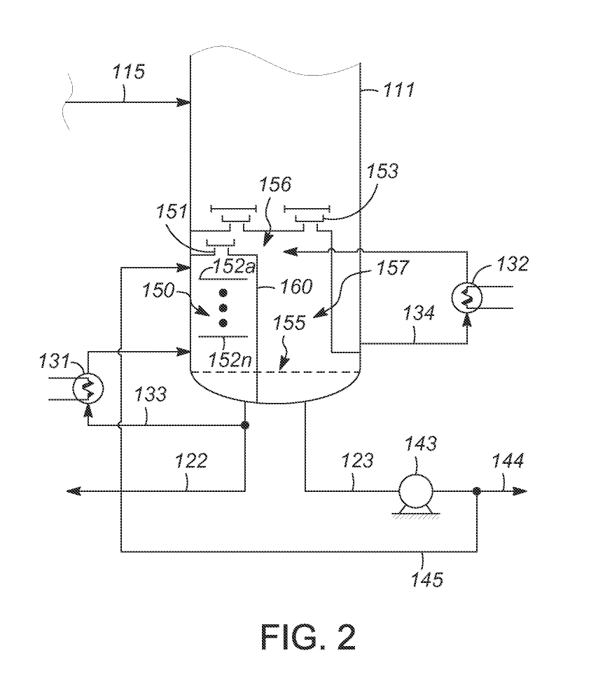

Desirably, embodiments of the present disclosure allow for the elimination of the need for a separate desorbent rerun column 20. Embodiments of the present disclosure incorporate the functionality of the desorbent rerun column into the raffinate column (e.g., raffinate column 11), as will be discussed in greater detail below in connection with the discussion of FIG. 2.

The preceding description of FIG. 1 has been provided in terms of the use of a single-component “heavy” (less volatile) desorbent in one specific separation. The adsorbent (stationary phase) and desorbent (mobile phase) are normally selected as a system for each specific separation. The use of multiple component desorbents is, however, very important in some separations. Sometimes the desorbent is less volatile than the extract and raffinate. For instance, the use of a mixture of a normal paraffin and an iso-paraffin, both several carbon numbers lighter than the feed, as a desorbent is commercially practiced in the separation of normal paraffins from a mixture of various other types of hydrocarbons.

Operating conditions for adsorption include, in general, a temperature range of from about 20° C. to about 250° C., such as from about 60° C. to about 200° C. Adsorption conditions also preferably include a pressure sufficient to maintain the process fluids in liquid phase, which may be from about atmospheric pressure to about 4.1×10⁶ Pa (about 600 psi). Desorption conditions generally include the same temperatures and pressures as used for adsorption conditions. Generally, an SMB process is operated with an A:F flow rate through the adsorption zone in the broad range of about 1:1 to about 5:1, where A is the volume rate of “circulation” of selective pore volume of the molecular sieve and F is the volumetric feed flow rate. The practice of embodiments of the present disclosure requires no significant variation in operating conditions, or adsorbent or desorbent composition within the adsorbent chambers. That is, the adsorbent preferably remains at the same temperature throughout the process.

Although much of the description herein is set in terms of use in an SMB process, embodiments of the present disclosure are applicable to other modes of performing adsorptive separation, such as a swing bed system employing one or more separate beds of adsorbent. As used herein, the term SMB is intended to refer broadly to the different systems that move the point of feed and desorbent insertion into adsorbent to simulate movement of the adsorbent.

Another variation in the performance of the process as depicted in FIG. 1 is the replacement of the rotary valve used as a desorbent flow control device with a manifold system of valves. Further variation is possible concerning which of the two streams enters which fractionation zone, which is determined primarily by practical engineering considerations.

As different separations are performed in the two separation columns, the mechanical details and equipment in the columns zones may differ. For instance, they may contain different types of fractionation trays, trays of the same type but at different spacing, or one fractionation column may contain or may be augmented by structured packing, as is known in the art.

The subject process is not believed to be limited to use with any particular form of adsorbent. The adsorbents employed in the process preferably include an inorganic oxide molecular sieve such as a type A, X, or Y zeolite or silicalite. Silicalite is a hydrophobic crystalline silica molecular sieve having intersecting bent-orthogonal channels formed with two cross-sectional geometries, 6 Å circular and 5.1-5.7 Å elliptical on the major axis. A wide number of adsorbents are known and a starting molecular sieve is often treated by ion exchange or steaming, etc., to adjust its adsorptive properties.

The active component of the adsorbents is normally used in the form of particle agglomerates having high physical strength and attrition resistance. The agglomerates contain the active adsorptive material dispersed in an amorphous, inorganic matrix or binder, having channels and cavities therein that enable fluid to access the adsorptive material. Methods for forming the crystalline powders into such agglomerates include the addition of an inorganic binder, generally a clay including a silicon dioxide and aluminum oxide, to a high purity adsorbent powder in a wet mixture. The binder aids in forming or agglomerating the crystalline particles. The blended clay-adsorbent mixture may be extruded into cylindrical pellets or formed into beads that are subsequently calcined in order to convert the clay to an amorphous binder of considerable mechanical strength. The adsorbent may also be bound into irregular shaped particles formed by spray drying or crushing of larger masses followed by size screening. The adsorbent particles may thus be in the form of extrudates, tablets, spheres, or granules having a desired particle range, preferably from about 16 to about 60 mesh (Standard U.S. Mesh) (about 1.9 mm to about 250 microns). Clays of the kaolin type, water permeable organic polymers, or silica are generally used as binders. The active molecular sieve component of the adsorbents will ordinarily be in the form of small crystals present in the adsorbent particles in amounts ranging from about 75 wt.-% to about 98 wt.-% of the particle based on a volatile-free composition. Volatile-free compositions are generally determined after the adsorbent has been calcined at 900° C. in order to drive off all volatile matter.

Those skilled in the art will appreciate that the performance of an adsorbent is often greatly influenced by a number of factors not related to its composition, such as operating conditions, feed stream composition, and the water content of the adsorbent. The optimum adsorbent composition and operating conditions for the process are therefore dependent upon a number of interrelated variables. One such variable is the water content of the adsorbent, which is expressed herein in terms of the recognized Loss on Ignition (LOI) test. In the LOI test, the volatile matter content of the zeolitic adsorbent is determined by the weight difference obtained before and after drying a sample of the adsorbent at a temperature of about 500° C. under an inert gas purge, such as nitrogen, for a period of time sufficient to achieve a constant weight. For the subject process, it is preferred that the water content of the adsorbent results in an LOI at about 900° C. of less than 7.0%, for example from about 0 wt.-% to about 4.0 wt.-%. The hydration level of the sieve has traditionally been maintained by the injection of water into the feed or desorbent streams.

An important characteristic of an adsorbent is the rate of exchange of the desorbent for the extract component of the feed mixture materials or, in other words, the relative rate of desorption of the extract component. This characteristic relates directly to the amount of desorbent material that must be employed in the process to recover the extract component from the adsorbent. Faster rates of exchange reduce the amount of desorbent material needed to remove the extract component, and therefore, permit a reduction in the operating cost of the process. With faster rates of exchange, less desorbent material has to be pumped through the process and separated from the extract stream for reuse in the process. Exchange rates are often temperature dependent. In one example, desorbent materials should a selectivity equal to about 1 or slightly less than about 1 with respect to all extract components, such that all of the extract components can be desorbed as a class with reasonable flow rates of desorbent material, and such that extract components can later displace desorbent material in a subsequent adsorption step.

In adsorptive separation processes, which are generally operated continuously at substantially constant pressures and a temperature that insures all compounds remain in the liquid phase, the desorbent material is selected to satisfy many criteria. First, the desorbent material should displace an extract component from the adsorbent with reasonable mass flow rates without itself being so strongly adsorbed as to unduly prevent an extract component from displacing the desorbent material in a following adsorption cycle. Expressed in terms of the selectivity, it is desirable that the adsorbent be more selective for all of the extract components with respect to a raffinate component than it is for the desorbent material with respect to a raffinate component. Secondly, desorbent materials should be compatible with the particular adsorbent and the particular feed mixture. More specifically, they should not reduce or destroy the capacity of the adsorbent or selectivity of the adsorbent for an extract component with respect to a raffinate component. Additionally, desorbent materials should not chemically react with or cause a chemical reaction of either an extract component or a raffinate component. Both the extract stream and the raffinate stream are typically removed from the adsorbent void volume in admixture with desorbent material and any chemical reaction involving a desorbent material and an extract component or a raffinate component or both would complicate or prevent product recovery. The desorbent should also be easily separated from the extract and raffinate components, as by fractionation. Finally, desorbent materials should be readily available and reasonable in cost. With proper attention to desorbent purity, sieve hydration level and adsorbent selection, the ratio of flow rates of desorbent and feed is often below about 1:1.

Reference will now be directed to FIG. 2, which depicts an exemplary “combined” raffinate column 111 (note that certain reference numerals have been incremented by 100 in FIG. 2), which combines the functionality of the raffinate column 11 of FIG. 1 and the desorbent rerun column 20 of FIG. 1. As in FIG. 1, a raffinate stream 15 provides the feed source to the combined raffinate column 111. The raffinate flows downward through trays 153 of the raffinate column portion 157 of the combined raffinate column 111. An external reboiler 132 may be incorporated to maintain the raffinate portion 157 of the combined column operating at a suitable temperature, the reboiled portion thereof passing through the reboiler 132 via line 134. Desorbent from the bottom of the raffinate portion 157 of the combined raffinate column 111 passes via line 123 and pump 143, whereafter a portion of the desorbent is passed to the desorbent rerun portion 150 of the combined column via line 145. The portion not passed thereto continues via line 144 to the rotary valve 2, as with line 23 in FIG. 1.

In this embodiment, the function of the desorbent rerun column is incorporated as a split-shell design inside the bottom of the raffinate column 111, in a space in the bottom of the column 111 at portion 150. This split-shell portion 150 consists of specially constructed trays 152a-152n to fit a chordal shape, the outer curved side conforming to the raffinate column 111 vessel wall, and the inner straight side conforming against the shell wall 160. In an alternate embodiment, the shape of the desorbent rerun section 150 could also be roughly rectangular, with one wall corresponding to the raffinate column shell.

The trays 152a-152n contain alternating down-comers, as with traditional distillation column trays. The shell wall 160 between the raffinate column bottom space 155 and the desorbent rerun section 150 are well insulated due to significant temperature differences between the two services. The top 151 of the desorbent rerun section 150 should communicate with the vapor space 156 of the raffinate column 111 in the manner of a chimney through a blind tray. The chimney 151 should have a top cover to prevent liquid from above entering the inner column 150. The desorbent rerun section bottom liquid can be reboiled by an external heat source in an external reboiler 131, via line 133. Heat to the reboiler 131 can be set to maintain a liquid level in the bottom of the desorbent rerun section. The temperature of the bottom of the desorbent rerun section 150 can be monitored, and heavy hydrocarbons can be removed via a small positive displacement pump (not shown) and line 122 as needed to maintain temperatures below an acceptable maximum.

The combined raffinate column 111 in the embodiments described herein are larger than the traditional desorbent rerun column (i.e., column 20 in FIG. 1), since the bottom of the combined raffinate column 111 is used herein as surge volume for the desorbent inventory, as noted above with regard to FIG. 1. The desorbent rerun section 150 of the split-shell column 111, which separates heavy hydrocarbons from the desorbent, can be located inside the combined raffinate column 111 bottom section. A raffinate column bottom stream from the discharge of the raffinate column portion 157 is introduced on the top tray 152a of the desorbent rerun section 150, with desorbent leaving the top of the inner column as vapor to return to the vapor space 156 of the bottom of the raffinate column portion 157, and the heavy hydrocarbon draw-off from the bottom of the desorbent rerun column portion 150 exiting via a nozzle in the shell to a reboiler 131. Reboiler vapors and liquid return to the column via a second nozzle (via line 133). A net heavy hydrocarbon stream can be pumped from the reboiler inlet line via line 122 and removed from the system.

The disclosed combination of the raffinate column and the desorbent rerun column into a single apparatus can overcome several drawbacks of the prior art. First, it is known that heavy hydrocarbons in the desorbent can be permanent poisons for the adsorbent. Heavies (i.e., C₉+ hydrocarbons) can reduce assembly performance, adsorbent life, and in severe cases can lead to shutdown/unload/ reload with new adsorbent with significant downtime and revenue losses. Removal of heavies from the recycle desorbent can maintain optimal assembly performance and adsorbent life. Having the removal of heavies from the recycle desorbent as an integral part of the raffinate column will ensure that the stripping operation of the recycle desorbent will not be bypassed. In another instance, removing recycled heavy hydrocarbons in the desorbent as an integral part of the raffinate column eases the operational guidelines and reduces the complexity of converting prior art designs that employed batch operation and multiple equipment operational changes. Further, the total capital cost and plot space of the overall assembly will be lower due to the elimination of the separate desorbent rerun column system. Still further, the design of the various embodiments herein realize an energy utilization benefit since the heat loss across separate columns is eliminated.

While at least one exemplary embodiment has been presented in the foregoing detailed description of the inventive subject matter, it should be appreciated that a vast number of variations exist. It should also be appreciated that the exemplary embodiment or exemplary embodiments are only examples, and are not intended to limit the scope, applicability, or configuration of the inventive subject matter in any way. Rather, the foregoing detailed description will provide those skilled in the art with a convenient road map for implementing an exemplary embodiment of the inventive subject matter. It is understood that various changes may be made in the function and arrangement of elements described in an exemplary embodiment without departing from the scope of the inventive subject matter as set forth in the appended claims.

1. A split-shell column comprising:
a raffinate column portion for separating a raffinate material from a desorbent material; and
a desorbent rerun column portion for separating heavy contaminants from the desorbent material,
wherein a feed to the desorbent rerun column portion is provided from the desorbent material in the raffinate column, and
wherein the desorbent rerun column portion occupies a portion of a lower end of the split-shell column and is thermally separated from the raffinate column portion.

2. The split-shell column of claim 1, wherein the raffinate material is a mixture comprising ortho- and meta-xylene.

3. The split-shell column of claim 2, wherein the desorbent material is relatively less volatile than the raffinate material.

4. The split-shell column of claim 1, wherein the raffinate column portion extends from a bottom of the split-shell column to a top of the split-shell column.

5. The split-shell column of claim 4, wherein the desorbent rerun column portion extends from the bottom of the split-shell column but does not extend to the top of the split-shell column.

6. The split-shell column of claim 5, wherein the desorbent rerun column portion occupies either a chordal-shaped or rectangular portion in a bottom portion of the split-shell column.

7. The split-shell column of claim 6, wherein the desorbent rerun column portion comprises a plurality of chordal-shaped or rectangular trays disposed therein.

8. The split-shell column of claim 7, wherein the chordal-shaped or rectangular trays are curved on one side to correspond with a shape of the split-shell column and are straight on another side to correspond with a shape of a dividing wall between the desorbent rerun column portion and the raffinate column portion.

9. The split-shell column of claim 8, wherein the dividing wall is insulated.

10. The split-shell column of claim 1, wherein the desorbent rerun column portion comprises a chimney disposed through a blind tray.

11. The split-shell column of claim 10, wherein vapor in the desorbent rerun column portion communicates with the raffinate column portion via the chimney disposed through the blind tray, and wherein liquid in the raffinate column portion is prevent from entry into the desorbent rerun column portion via the chimney disposed through the blind tray.

12. The split-shell column of claim 1, wherein the heavy contaminants comprise C₉+hydrocarbons.

13. The split-shell column of claim 1, wherein the desorbent rerun column portion comprises a reboiler and wherein the raffinate column portion comprises a reboiler.

14. The split-shell column of claim 1, wherein a first portion of the desorbent separated in the raffinate column portion is directed to the desorbent rerun column portion and wherein a second portion of the desorbent separated in the raffinate column portion is directed away from the split-shell column.

15. The split-shell column of claim 1, wherein the desorbent material separated in the desorbent rerun column is directed back into the raffinate column portion.

16. The split-shell column of claim 1, wherein the heavy contaminants separated in the desorbent rerun column portion are directed away from the split-shell column.

17. A method for separating hydrocarbon mixtures, the method comprising the steps of:
directing a raffinate stream into a split-shell column, the raffinate stream comprising a raffinate material and a desorbent material, the split-shell column comprising a raffinate column portion and a desorbent rerun column portion; separating the raffinate material from the desorbent material in the raffinate column portion of the split-shell column;
directing a first portion of the desorbent material separated in the raffinate column portion to the desorbent rerun column portion;
separating the desorbent material into decontaminated desorbent material and heavy contaminants; and
directing the decontaminated desorbent material back into the raffinate column portion.

18. The method of claim 17, further comprising directing a second portion of the desorbent material separated in the raffinate column portion away from the split-shell column.

19. The method of claim 18, further comprising directing the heavy contaminants separated in the desorbent rerun column away from the split-shell column.

20. A split-shell column comprising:
a raffinate column portion for separating a raffinate material from a desorbent material, wherein the raffinate column portion extends from a bottom of the split-shell column to a top of the split-shell column. a desorbent rerun column portion for separating heavy contaminants from the desorbent material, wherein the desorbent rerun column portion extends from the bottom of the split-shell column but does not extend to the top of the split-shell column,
wherein a feed to the desorbent rerun column portion is provided from the desorbent material separated in the raffinate column,
wherein the desorbent rerun column portion occupies a portion of a lower end of the split-shell column and is thermally separated from the raffinate column portion with an insulated dividing wall,
wherein the desorbent rerun column portion comprises a chimney disposed through a blind tray, and
wherein the desorbent material separated in the desorbent rerun column portion is directed back into the raffinate column portion through the chimney disposed through the blind tray.
